(19) United States
(12) Patent Application Publication
(10) Pub. No.: US 2006/0089616 A1
(43) Pub. Date: Apr. 27, 2006

(54) Absorbent article having dual pattern bonding

(75) Inventors: Tom R Belau, Neenah, WI (US); Melissa C Putzer, Oshkosh, WI (US); John F Condron, Neenah, WI (US); Gregory L Malchow, Oshkosh, WI (US); Charles P Keuhn JR., Neenah, WI (US); Thomas D Ehlert, Neenah, WI (US); Paul J Datta, Appleton, WI (US); Barbara A Gossen, Oshkosh, WI (US); Patrick R Lord, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark World, Inc., Neenah, WI (US)

(21) Appl. No.: 10/524,718

(22) PCT Filed: Aug. 20, 2003
(86) PCT No.: PCT/US03/26183

§ 371 Date: Feb. 15, 2005

Related U.S. Application Data
(60) Provisional application No. 60/405,861, filed on Aug. 22, 2002.
(60) Provisional application No. 60/406,199, filed on Aug. 26, 2002.

(51) Int. Cl. A61F 13/15

(52) U.S. Cl. 604/389; 604/385.11

(57) ABSTRACT

The present invention relates to improvements in bonding patterns for the attachment of components of disposable absorbent articles. For example, in order to provide attachment of an ear portion to a longitudinal side of the chassis of an article, there may be an attachment area. The attachment area may include an overlapping area of chassis material(s) (outer cover and/or liner materials) and ear portion material. The overlapping areas of chassis material and ear portion material are bonded together in such a way as to include a variegated bond pattern and a uniform bond pattern. The bonding patterns of the invention may also be used to form the side seams and passive side bonds for articles that are donned as pants.

This patent application is a non-provisional patent application claiming priority to U.S. provisional patent application Ser. No. 60/406,199 filed on Aug. 26, 2002.

### BACKGROUND OF THE INVENTION

Disposable absorbent articles such as infant diapers, diaper-pants, training pants, feminine care pads and adult incontinence pads/garments are complex products. These types of articles include multiple components, are made on high speed production lines, need to be cost affordable and most importantly, must perform their intended function. In order to be of the quality expected by consumers, individual articles need to include a complete set of the individual components (e.g. an absorbent pad, pair of fasteners, pair of leg elastics, pair of containment flaps, etc.). Manufacturing systems have been developed that bring all of the components together in one “web” of materials from which individual articles are formed. The quality of the individual articles is controlled so that each article has the intended components and the components are properly attached to and aligned with each other, with some tolerance for manufacturing variability.

Disposable absorbent articles are typically constructed of multiple nonwoven material components. For example, a disposable absorbent article may include a bodyside liner (also known as a topsheet) that contacts the wearer's skin in use, an absorbent core and an outer cover (also known as a backsheet) that is typically liquid impermeable. In addition, disposable absorbent articles may include components such as leg elastics, waist elastics, containment flaps, front waist ear portions, back waist ear portions and fastening systems for improving the fit and containment of the articles. In order to form the finished article, the various components are placed in desired relationship to each other and, where necessary, the components are attached to each other. While adhesive may be used to provide the attachment between some components, it may be desirable to attach other components using other bonding techniques, such as ultrasonic bonding. For example, it may be desirable to use ultrasonic bonding to attach components to the ”chassis” of the article. The “chassis” of a disposable absorbent article typically refers to the combination of the bodyside liner, absorbent core and outer cover components. The chassis of an article may include two longitudinal edges, that generally define the length of an article, and two lateral edges, that generally define the waist edges and width of an article.

Ear portions are components that may be attached to the longitudinal edges of an article's chassis. Presently-available infant diapers typically include “ears” that are attached near the waist edges of the article. The term “ear” provides a representative description of the component because, in part, the ear portion extends generally perpendicular to the longitudinal centerline of the article. The ear portion may Include a tape fastener or a mechanical fastener. When the ear portion Includes a fastener, the ear portion enables the fastener to be provided in a laterally outward position so that the article is capable of providing a wider range of fit. Typically, the fastener is engaged with the waist region opposite the waist region where the ear portion is attached. The ear portion may include a stretchable material in order to increase the fit range of the article. Additionally, the ear portion may be shaped and the ear portion may provide part of the leg opening that is formed when the article is worn. Presently-available infant diapers may include a pair of ear portions attached to the front waist region of the diaper and a pair of ear portions attached to the back waist region of the diaper. In addition to increasing the fit range of the article, ear portions may be used to improve placement of the article about the wearer and to improve how well the article fits the wearer. For example, front waist ear portions may aid a caregiver's positioning of a diaper on the child.

When the ear portion includes a fastener, the ear portion may experience significant stress forces during application and wear of the article. For example, a caregiver may pull on the ear portion in order to wrap the ear portion around the side of the child and in order to engage the fastener with the opposite waist region. Consequently, it is desirable for the ear portion to be securely attached to the chassis of the article. If the attachment is not secure, the ear portion may tear or be pulled entirely off of the chassis resulting in the article being unusable or having poor fit.

In addition to ear portions, there are additional components of disposable absorbent articles that may benefit from attachment to the chassis or to each other provided by ultrasonic bonds. For example, with some product forms, the article may include both front ear portions and back ear portions and it may be desirable for the front ear portions to be attached to the back ear portions by ultrasonic bonds. “Diaper-pants” are disposable absorbent articles that have the absorbent capacity of typical infant diapers, but that may be put on the wearer like a pair of underpants. Diaper-pants may be a desirable product form for young children who are able to walk and who resist laying down for changing, but who are not yet toilet-trained. With a diaper-pant article, a back ear portion may be bonded to a front ear portion to form a side bonded area. Another example of a pant-like product form is training pant disposable absorbent articles. Presently-available training pant articles include front and back side panels attached to the longitudinal edges of the pant chassis and attached to each other to form side seams.

Desirably, one of the functions of ultrasonic bonding attachment is to provide sturdy and durable attachment of components to the chassis of articles and to each other. Sturdy and durable attachment is necessary for the components to withstand the forces experienced during application and wear of the articles. Another function of ultrasonic bonding attachment is the bonding together of the component materials in such a way that the materials are not readily detachable in the area of the bonds. In other words, it is desirable for the bonds to be formed in such a way that the bonded area does not act as a perforation for materials to become separated from each other.

In very simplified terms, ultrasonic bonding involves the vibration of a “horn” device at ultrasonic frequencies which, in combination with an “anvil” (configured in a desired bond pattern), causes localized heating and bonding of substrate material(s) positioned between the horn and the anvil. Various ultrasonic bond patterns may be achieved through the selection of an appropriate anvil. Additionally, anvils can be selected to provide more than one ultrasonic bond pattern at the same time. Conventional ultrasonic bond patterns position adjacent bond elements in uniform straight lines and crossing rows. The design of these conventional bond patterns have evolved as a replacement for adhesive bonding. Because bond patterns have been selected to replace adhesive bonding, the bond patterns have not necessarily been optimized.

For example, adhesive bonding usually causes minimal or no damage to the base material and therefore, from a bond strength perspective, more adhesive per length or area is generally better. However, in contrast, more ultrasonic bonding per length or area, may cause degradation of the material or materials being bonded together. Consequently, bond strength may be decreased. In addition to the amount of bonding over a given length or area, there are other aspects of the translation from adhesive to ultrasonic bonding that have not been optimized. When using adhesive to bond two materials with minimal overlap to each other, if the adhesive extends beyond the area of overlap of the materials, there may be processing and product problems. Specifically, there may be adhering of materials to the processing machine causing product to become wrapped around drive rollers, or causing exposed adhesive on the product which can cause the product to stick to itself, products to stick to one another, or product to stick to the user. Consequently, narrow slot adhesive processes may be desirable for bonding materials with minimal areas of overlap. If the materials being attached to each other are inherently stretchable, the adhesive may tie up the stretchability of the materials. Traditionally, ultrasonic bond patterns have been narrow, tightly packed rows of bond points. Tightly packed rows most closely duplicate the narrow adhesive slot coat, therefore minimizing the degree to which stretch of the materials is reduced. Additionally, tightly packed rows may be more process friendly because they minimize hopping and the narrow width prevents pressure variation from side to side.

While bond patterns have been selected to replicate the attachment area provided by adhesive attachment, the bond patterns have not been optimized for strength (e.g. minimal tearing of materials being bonded) or durability. Consequently, there remains a need in the area of disposable absorbent articles for bond patterns that provide improved attachment of components to the chassis of such articles. There also remains a need for bond patterns that attach one or more materials together with integrity without overly weakening or perforating the materials being attached.

### SUMMARY OF THE INVENTION

In one aspect, the present invention relates to the use of two or more bond patterns for the bonding of components, such as ear portions, to the chassis of an absorbent article. As described herein, absorbent articles typically include three primary components: a bodyside liner, an absorbent core and an outer cover. The “chassis” typically includes the outer cover, the bodyside liner or both the outer cover and the bodyside liner. Typically, the two-dimensional area of the absorbent core is somewhat less than the entire area of the chassis. The bodyside liner and outer cover materials are generally contiguous and together, they surround the absorbent core. Many absorbent articles have systems for the fastening of the article about a wearer. The fastening systems may include mechanical fasteners such as a hook material that engages a complementary loop material to join one waist region to the opposite waist region. It is possible for the hook material to be located directly on the chassis materials of the article. More typically however, the hook material is located on a back or front ear portion that is attached to the chassis. The most common configuration is for the hook material to be located on a back ear portion for engagement with a complementary loop material located in the front waist region of the outer cover. Placement of the hook material on a back ear portion that extends laterally outward from the chassis facilitates joining of the waist regions around a wearer of the article. The present invention includes the use of bond patterns to attach components, such as ear portions, to improve the integrity of attachment and to reduce tearing of bonded materials that may occur during use of the articles.

The present invention includes a disposable absorbent article that has a longitudinal direction and a lateral direction. The longitudinal direction may correspond to the “machine direction” of the article, which is the direction in which the article is manufactured. The lateral direction may be generally perpendicular to the longitudinal direction. Typically, the longitudinal dimension of the article is longer than the lateral dimension. The disposable absorbent article may also include an outer cover and the outer cover may have a longitudinal edge. The longitudinal edge may be generally aligned with the longitudinal direction of the article. The outer cover may also include an outer cover material. The outer cover material may be a laminate of a nonwoven material and a liquid impermeable film material. Either or both of the nonwoven material and the film material may be extensible, stretchable or elastic. The disposable absorbent article may also include an ear portion that extends laterally outward from the longitudinal edge of the outer cover. Therefore, the ear portion may extend in a direction that is generally perpendicular to the longitudinal direction of the article. The ear portion may include an ear portion material. The ear portion material may be extensible, stretchable or elastic. The disposable absorbent articles may also include an attachment area that may include an overlapping area of the outer cover material and the ear portion material. The attachment area may also include a variegated bond pattern and a uniform bond pattern.

Disposable absorbent articles such as infant diapers, diaper-pants and training pants typically include a front waist region, a back waist region and a crotch region that interconnects the front waist region and the back waist region. The ear portions may be attached to either or both of the front waist region and the back waist region. A combination of a variegated bond pattern and a uniform bond pattern may be used to attach the ear portion to the chassis of the absorbent article. The variegated bond pattern may include a plurality of individual bond points where the bond points are not all located equidistantly from each other. The uniform bond pattern may include a plurality of individual bond points where the bond points are located generally equidistant from each other. In a combination of a variegated bond pattern and a uniform bond pattern, the variegated bond pattern may be located laterally outward from the uniform bond pattern. When a force is applied to an ear portion (as may occur during use of the article), the bond points of the variegated bond pattern may preferentially fail before the bond points of the uniform bond pattern. Additionally, it is believed that the when an ear portion is pulled by a wearer or caregiver, the stress forces are absorbed as the bond points of the variegated bond pattern fail, resulting in force dissipation and a reduced stress force on the bond points of the uniform bond pattern. The bond points of the variegated bond pattern are believed to act as an energy absorber to prevent fracture of the bond points of the uniform bond pattern. The result of such d“dual” bond patterns is improved tear resistance of the ear portion by requiring fracture of both the variegated bond pattern and the uniform bond pattern for a failure to occur while also providing more complete bonding of the ear portion to the chassis materials. For absorbent articles like diaper-pants and training pants, the combination of the variegated bond pattern and the uniform bond pattern may also be used to form the attachment between the front and rear “side panels” of such articles.

The bond points of the bond patterns of the invention may be formed by ultrasonic bonding techniques, but they may also be formed by other bonding techniques known to those of skill in the art. Various characteristics of the individual bond points of both the variegated bond pattern and the uniform bond pattern have been identified as beneficial. For example, the individual bond points of the variegated bond pattern may have an area of from about 0.00017 square inches (in²) to about 0.0032 square inches (in²). The individual bond points of the uniform bond pattern may have the same ranges of areas. The individual bond points of both the variegated bond pattern and the uniform bond pattern may also have a bond angle of from about 5 to about 25 degrees. While the individual bond points of the two bond patterns may be similar with respect to area and bond angle, the individual bond points of the two bond patterns may have different thicknesses. For example, the variegated bond pattern may have a thickness and the uniform bond pattern may have a thickness where there is a normalized bond thickness differential between the two bond patterns of from about 3.7 to about 13.6 mm/g/cm² (millimeters/grams/square centimeters). As will be described in greater detail herein, the bond thickness differential is “normalized” to take into account the different possible basis weights of the materials being bonded together.

The variegated bond pattern and the uniform bond pattern may be located in close proximity to each other. For example, the variegated bond pattern and the uniform bond pattern may have a lateral spacing from each other of from about 2 mm (millimeters) to about 10 mm (millimeters). However, the two bond patterns may be spaced further apart depending on the particular article configuration. Additionally, the variegated bond pattern and the uniform bond pattern may have a combined width in the lateral direction of from about 8 to about 25 millimeters.

In addition to providing attachment of ear portions to the chassis and formation of passive side bonds, the combination of bond patterns of the invention may also be used to attach a fastener material to the laterally outward (or distal) edge of an ear portion. For example, a hook fastener material may be attached to the distal edge of a back ear portion by a combination of a variegated bond pattern and a uniform bond pattern. The ear portion may further include a fastener attachment area. The fastener attachment area may include an overlapping area of the ear portion material and a fastener material. The fastener material may be extensible, stretchable or elastic and may include an adhesive, hooks, buttons or another suitable fastening material. In addition to a combination of a variegated bond pattern and a uniform bond pattern, the fastener attachment area may alternatively include a low density bond pattern and a high density bond pattern. The difference in “density” between two such bond patterns may be in terms of the percentage of bonded area. For example, the low density bond pattern may have a percent bond area of from about 9% to about 18% and the high density bond pattern may have a percent bond area of about 25%.

In another aspect, the present invention relates to a disposable absorbent article that includes a longitudinal direction and a lateral direction. The disposable absorbent article may also include a bodyside liner that has a longitudinal edge and includes a bodyside liner material. The bodyside liner material may be extensible, stretchable or elastic. The disposable absorbent article may include an ear portion where the ear portion extends laterally outward from the longitudinal edge of the bodyside liner. The ear portion may include an ear portion material that may be extensible, stretchable or elastic. The absorbent article may also include an attachment area where the attachment area includes an overlapping area of the bodyside liner material and the ear portion material. The attachment area may also include a variegated bond pattern and a uniform bond pattern. Both the variegated bond pattern and the uniform bond pattern may include individual bond points having areas and bond angles similar to those already described herein. Further, the variegated bond pattern and the uniform bond pattern may be spaced apart from each other and have a combined width in the lateral direction as already described herein. The variegated bond pattern may be located laterally outward from the uniform bond pattern. The individual bond points of the variegated bond pattern may have a thickness and the individual bond points of the uniform bond pattern may have a different thickness where there is a normalized bond thickness differential of from about 3.7 to about 13.6 mm/g/cm². The ear portion may also include a fastener attachment area that includes an overlapping area of ear portion material and fastener material. The fastener attachment area may also include a low density bond pattern (which may have a percent bond area of about 9% to about 18%) and a high density bond pattern (which may have a percent bond area of about 25%).

In another aspect, the present invention relates to a disposable absorbent article similar to those already described, except the attachment area may include an overlapping area of an outer cover material, a bodyside liner material and an ear portion material. The materials may be fastened together by a combination of a variegated bond pattern and a uniform bond pattern. In a different aspect, the present invention relates to a disposable absorbent article including a longitudinal direction and a lateral direction. The disposable absorbent article may also include a chassis where the chassis includes a front waist region, a back waist region and a crotch region intermediate the front waist region and the back waist region. The absorbent article may include a back ear portion that is attached to the back waist region and the absorbent article may include a front ear portion that is attached to the front waist region. The absorbent article may further include a side bond between the back ear portion and the front ear portion. The side bond may include a variegated bond pattern and an uniform bond pattern as described herein.

The present invention also relates to a similar disposable absorbent article including a side bond between a back ear portion and a front ear portion, but where the side bond may include a short distance bond pattern and a long distance bond pattern. The short distance bond pattern and the long distance bond pattern may differ from each other with respect to the spacing between the individual bond points. For example, the individual bond points of the long distance bond pattern may be spaced apart from each other by at least 0.5 inches. The individual bond points of the short distance bond pattern may be spaced apart from each other by less than 0.5 inches. Additionally, the short distance bond pattern may have a smaller bonded area than the long distance bond pattern. The individual bond points of the long distance bond pattern may be larger than the individual bond points of the short distance bond pattern. For example, the individual bond points of the long distance bond pattern may have an area of from about 1.7 to about 28 square millimeters (mm²) and the individual bond points of the short distance bond pattern may have an area of from about 0.75 to about 3 square millimeters (mm²). The individual bond points of the long distance bond pattern and the individual bond points of the short distance bond pattern may also differ with respect to their thickness.

These aspects and additional aspects of the invention will be described in greater detail herein. Further, it is to be understood that both the foregoing general description and the following detailed description are exemplary and are intended to provide further explanation of the invention claimed. The accompanying drawings, that are incorporated in and constitute part of this specification, are included to illustrate and provide a further understanding of the disposable absorbent articles of the invention. Together with the description, the drawings serve to explain various aspects of the invention.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1 representatively shows a perspective view of an example of a disposable absorbent article (an infant diaper) of the present invention;

FIG. 2 representatively shows a plan view of the disposable absorbent article of FIG. 1 in an unfastened, stretched and laid flat condition with the surface of the article which contacts the wearer's skin facing the viewer and with portions of the article partially cut away to show the underlying features;

FIG. 3 representatively shows a perspective view of another example of a disposable absorbent article (a diaper-pant) of the present invention;

FIG. 4 representatively shows a plan view of the disposable absorbent article of FIG. 3 in an unfastened, stretched and laid flat condition with the surface of the article which contacts the wearer's skin facing the viewer and with portions of the article partially cut away to show the underlying features;

FIG. 5 representatively shows a plan view of an example of an attachment area between a back ear portion and the longitudinal side edge of the chassis of a disposable absorbent article including a variegated bond pattern and a uniform bond pattern;

FIG. 6 representatively shows a plan view of an example of a variegated bond pattern adjacent a uniform bond pattern;

FIG. 7 representatively shows a plan view of another example of a variegated bond pattern adjacent a uniform bond pattern;

FIG. 8 representatively shows a plan view of an example of a combination of a short distance bond pattern and a long distance bond pattern of the invention that may be used for forming a passive side bond on a diaper-pant type of article;

FIG. 9 representatively shows a plan view of an example of a combination of a low density bond pattern and a high density bond pattern of the invention that may be used for providing attachment of a fastener to an ear portion;

FIG. 10 representatively shows a photograph of a bond pattern (at a magnification of 5.3×) that was used to measure bond strengths as described in more detail herein; and

FIG. 11 representatively shows a photograph of the digital thickness measurement gauge and associated apparatus used to measure the thickness of bond points.

### DETAILED DESCRIPTION OF THE INVENTION

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of Its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The present invention is directed to solving problems related to absorbent articles including the durability and integrity with which components, such as ear portions, are attached to the chassis of the articles. Additionally, the present invention is directed to improving the durability and integrity with which components, such as ear portions, are attached to each other. This detailed description of the present invention will include a description of a representative absorbent article including the various components of such articles. The description of the representative absorbent article will also include a description of the features encompassed by the present invention.

Representative Absorbent Article

The absorbent articles of the present invention will be described in terms of a disposable diaper article and in terms of a diaper-pant that is adapted to be worn by infants about the lower torso. It is understood that the features of the present invention are equally adaptable for other types of absorbent articles such as adult incontinence pads, adult incontinence garments, training pants, disposable swim pants and feminine hygiene pads.

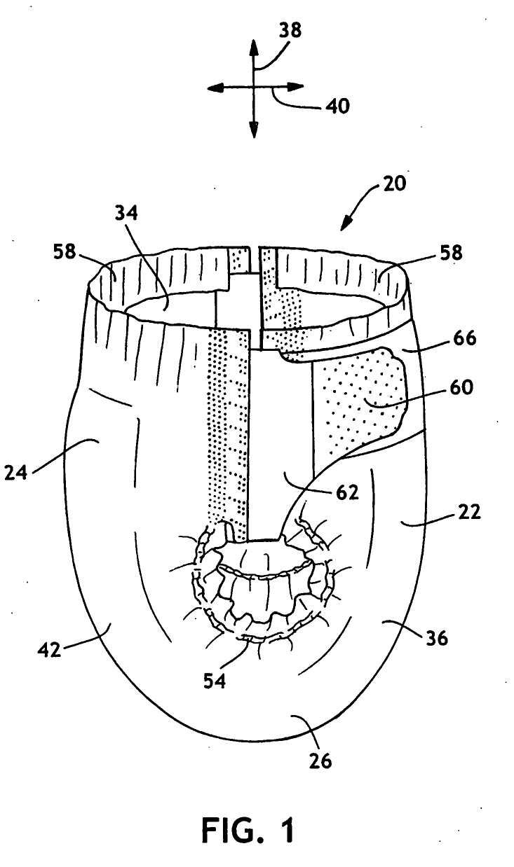

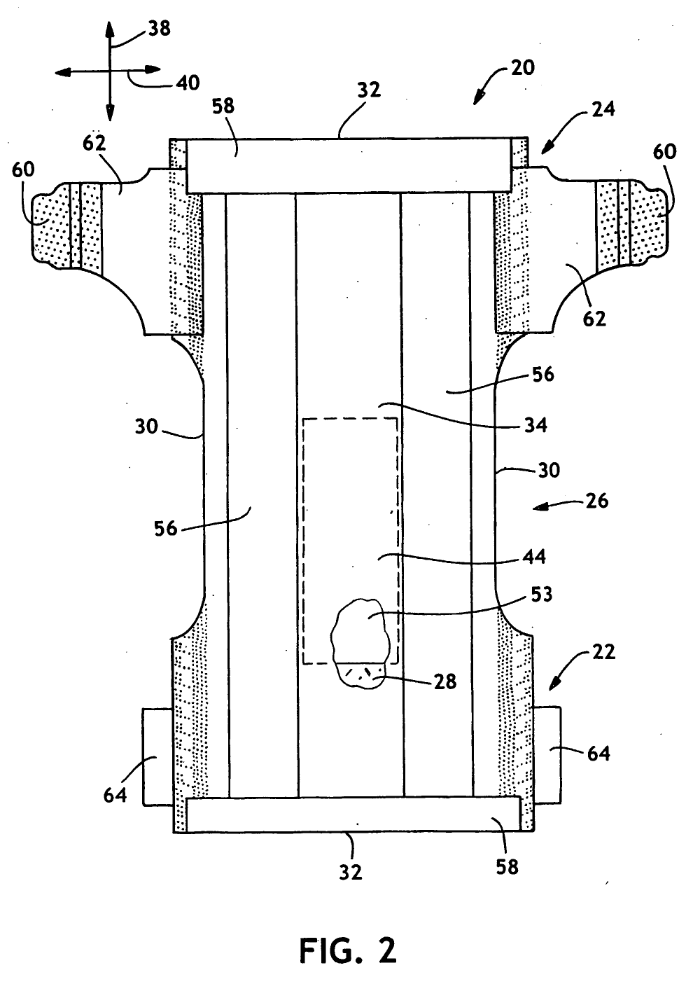

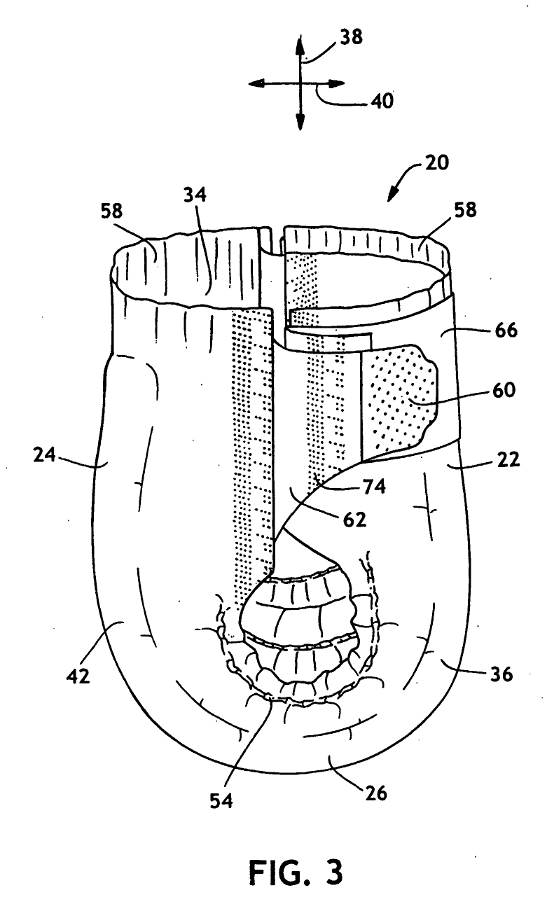

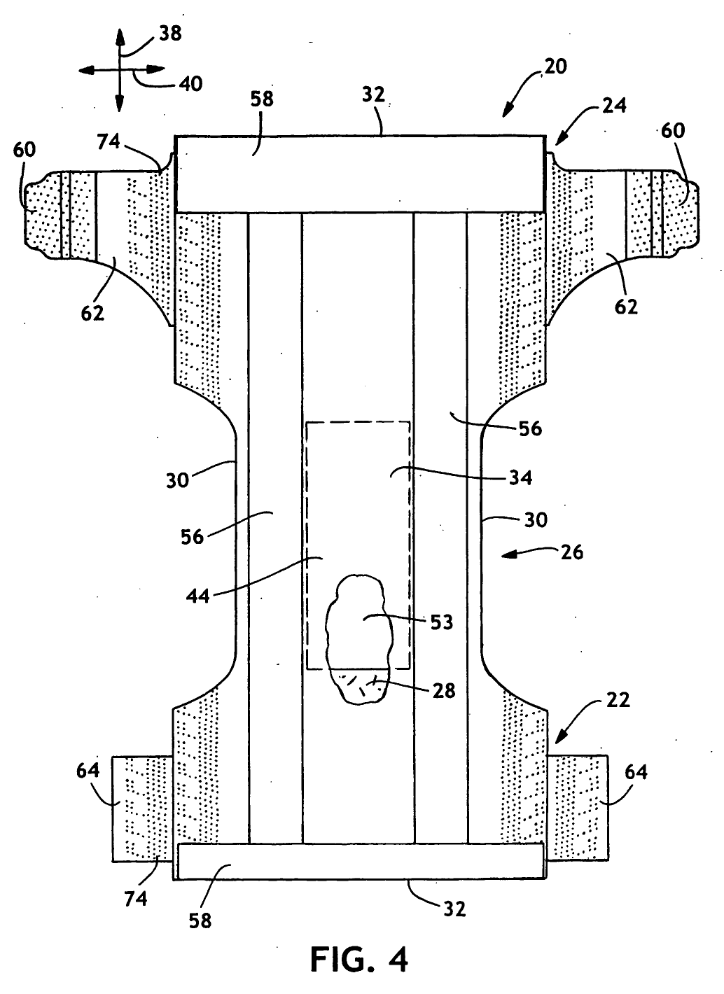

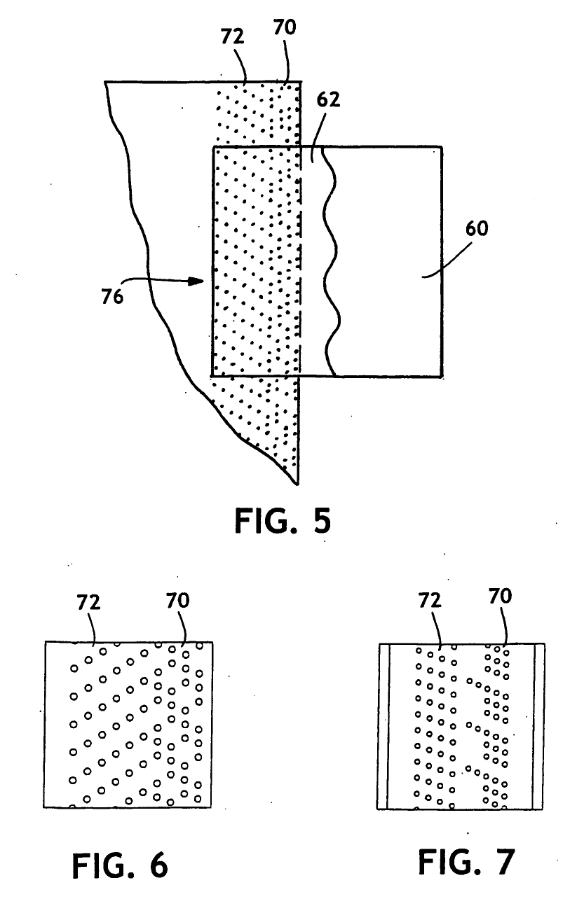

FIG. 1 representatively illustrates an example of a refastenable disposable diaper, as generally indicated at 20, of the present invention. FIG. 2 representatively illustrates the refastenable diaper of FIG. 1 in an unfastened, stretched and laid flat configuration with the surface of the diaper adapted to contact the wearer's skin facing the viewer and with portions of the diaper partially cut away to show the underlying features. FIG. 3 representatively illustrates another example of an absorbent article of the invention, a diaper-pant generally indicated at 20. FIG. 4 representatively illustrates the prefastened diaper-pant of FIG. 3 in an unfastened, stretched and laid flat configuration with the surface of the diaper-pant adapted to contact the wearer's skin facing the viewer and with portions of the diaper-pant partially cut away to show the underlying features. As illustrated in FIG. 2 and FIG. 4, the diaper/diaper-pant 20 defines a front waist region 22, a back waist region 24, a crotch region 26 that extends between and connects the front and back waist regions 22 and 24, a longitudinal direction 38 and a lateral direction 40. The front waist region 22 includes the portion of the diaper/diaper-pant 20 that, when worn, is positioned on the front of the wearer while the back waist region 24 includes the portion of the diaper/diaper-pant 20 that, when worn, is positioned on the back of the wearer. The crotch region 26 of the diaper/diaper-pant 20 includes the portion of the diaper/diaper-pant 20 that, when worn, is positioned between the legs of the wearer and covers the lower torso of the wearer.

The diaper/diaper-pant 20 defines a pair of laterally opposed side edges 30, a pair of longitudinally opposed waist edges 32, an interior surface 34 that is configured to contact the wearer, and an exterior surface 36 opposite the interior surface 34 that is configured to contact the wearer's clothing in use. The illustrated diaper/diaper-pant 20 also includes a substantially liquid impermeable outer cover 42 and a liquid permeable bodyside liner 44 that can be connected to the outer cover 42 in a superposed relation. An absorbent core 28 is located between the outer cover 42 and the bodyside liner 44. The laterally opposed side edges 30 of the diaper/diaper-pant 20 are generally defined by the side edges of the outer cover 42 that further define leg openings that may be curvilinear. The waist edges 32 of the diaper/diaper-pant 20 are generally defined by the waist edges of the outer cover 42 and define a waist opening that is configured to encircle the waist of the wearer when worm. The absorbent core 28 is configured to contain and/or absorb body exudates discharged from the wearer. The diaper/diaper-pant 20 may further include leg elastics 54, containment flaps 56 and waist elastics 58 as are known to those skilled in the art. It should be recognized that individual components of the diaper/diaper-pant 20 may be optional depending upon the intended use of the diaper/diaper-pant 20.

The diaper/diaper-pant 20 may further include refastenable mechanical fasteners 60. The mechanical fasteners 60 releasably engage the opposed side edges 30 of the diaper/diaper-pant 20 in the opposite waist regions. The mechanical fasteners 60 can include a variety of materials and surfaces known for mechanical engagement such as buttons, pins, snaps, adhesive tape fasteners, cohesives, mushroom-and-loop fasteners and hook and loop fasteners. Further, the disposable diaper/diaper-pant 20 may include an attachment panel 66 located on the front or back waist region 22 and 24, opposite the fasteners 60 to which the fasteners 60 can be releasably engaged during use of the diaper/diaper-pant 20.

The diaper/diaper-pant 20 may be of various suitable shapes. For example, in the unfastened configuration as illustrated in FIG. 2 and FIG. 4, the diaper/diaper-pant 20 may have an overall rectangular shape, T-shape or an approximately hourglass shape. In the shown embodiments, the diaper/diaper-pant 20 has a generally I-shape in an unfastened configuration.

The various components of the diaper/diaper-pant 20 are integrally assembled together employing various types of suitable attachment means, such as adhesive, sonic and thermal bonds or combinations thereof. In the shown embodiments, for example, the outer cover 42 and bodyside liner 44 are assembled to each other and to the absorbent core 28 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Alternatively, the absorbent core 28 may be connected to the outer cover 42 using conventional fasteners such as buttons, hook and loop type fasteners, adhesive tape fasteners, and the like. The other components of the diaper/diaper-pant 20 may be suitably connected together using similar means. Similarly, other diaper components, such as the elastic members 54 and 58 and the mechanical fasteners 60, may be assembled into the diaper/diaper-pant 20 article by employing the above-identified attachment mechanisms. Desirably, the majority of the diaper components are assembled together using ultrasonic bonding techniques for reduced manufacturing cost.

The outer cover 42 of the diaper/diaper-pant 20, as representatively illustrated in FIG. 1 and FIG. 3, may suitably be composed of a material which is either liquid permeable 20 or liquid impermeable. It is generally preferred that the outer cover 42 be formed from a material that is substantially impermeable to liquids. A typical outer cover can be manufactured from a thin plastic film or other flexible liquid-impermeable material. For example, the outer cover 42 may be formed from a polyethylene film having a thickness of from about 0.013 millimeter (0.5 mil) to about 0.051 millimeter (2.0 mils). The materials of the outer cover 42 can be thermally or adhesively laminated together. Suitable laminate adhesives, which can be applied continuously or intermittently as beads, a spray, parallel swirls, or the like, can be obtained from Bostik-Findley, Inc., of Wauwatosa, Wis., U.S.A., or from National Starch and Chemical Company, Bridgewater, N.J., U.S.A. If it is desired to present the outer cover 42 with a more clothlike feeling, the outer cover 42 may be formed from a polyolefin film having a nonwoven web laminated to the exterior surface thereof, such as a spunbond web of polyolefin fibers. For example, a stretch-thinned polypropylene film having a thickness of about 0.015 millimeter (0.6 mil) may be thermally laminated thereto a spunbond web of polypropylene fibers. The polypropylene fibers may have a fiber diameter of about 15 to 20 microns, which nonwoven web has a basis weight of about 17 grams per square meter (0.5 ounce per square yard). The outer cover 42 may include bicomponent fibers such as polyethylene/polypropylene bicomponent fibers. Methods of forming such clothlike outer covers are known to those skilled in the art. The outer cover 42 may also be an extensible outer cover such as the outer covers described in U.S. Pat. No. 6,552,245 issued on Apr. 22, 2003 to Roessler et al. The outer cover 42 may also be a biaxially stretchable outer cover such as the outer covers described in U.S. patent application Ser. No. 09/698,517 filed on Oct. 27, 2000 by Vukos et al.

The outer cover 42 may be formed of a woven or nonwoven fibrous web layer which has been totally or partially constructed or treated to impart a desired level of liquid impermeability to selected regions that are adjacent or proximate the absorbent core 28. Still further, the outer cover 42 may optionally be composed of a micro-porous “breathable” material which permits vapors to escape from the absorbent core 28 while still preventing liquid exudates from passing through the outer cover 42. For example, the outer cover 42 may include a vapor permeable non-woven facing layer laminated to a micro-porous film. Suitable “breathable” outer cover materials are described in U.S. Pat. No. 5,695,868 issued Dec. 9, 1997 to McCormack et al. and U.S. Pat. No. 5,843,056 issued Dec. 1, 1998 to Good et al., the descriptions of which are hereby incorporated by reference. Still further, the outer cover 42 may also be an elastomeric material such as a stretch-thermal laminate (STL), neck-bonded laminate (NBL), or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. The outer cover 42 can also be embossed or otherwise provided with a matte finish to provide a more aesthetically pleasing appearance.

In order to reduce the perception that the outer cover 42 feels damp or clammy, the diapers/diaper-pants 20 of the invention may include a spacer or ventilation layer (not shown in Figures) between the garment-facing surface of the absorbent core 28 and the outer cover 42. The ventilation layer may include one or more nonwoven materials, for example a spunbond-meltblown-spunbond nonwoven material.

The representative absorbent articles of the invention include a bodyside liner 44 in superimposed relation to the outer cover 42. The bodyside liner 44, as representatively illustrated in FIG. 2 and FIG. 4, suitably presents a bodyfacing surface that is compliant, soft feeling, and nonirritating to the wearer's skin. Further, the bodyside liner 44 may be less hydrophilic than the absorbent core 28, to present a relatively dry surface to the wearer, and may be sufficiently porous to be liquid permeable, permitting liquid to readily penetrate through its thickness. A suitable bodyside liner 44 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 44 is suitably employed to help isolate the wearer's skin from liquids held in the absorbent 28. The bodyside liner 44 can also be made from extensible materials as are described in U.S. Pat. No. 6,552,245 issued on Apr. 22, 2003 to Roessler et al. The bodyside liner 44 can also be made from biaxially stretchable materials as are described in U.S. patent application Ser. No. 09/698,517 filed on Oct. 27, 2000 by Vukos et al.

Various woven and nonwoven fabrics can be used for the bodyside liner 44. For example, the bodyside liner may be composed of a meltblown or spunbond web of polyolefin fibers. The bodyside liner 44 may also be a bonded-carded web composed of natural and/or synthetic fibers. The bodyside liner 44 may be composed of a substantially hydrophobic material, and the hydrophobic material may optionally be treated with a surfactant or otherwise processed to impart a desired level of wettability and hydrophilicity. In a particular embodiment of the present invention, the bodyside liner 44 is made from a nonwoven, spunbond, polypropylene fabric composed of fibers having a fiber diameter of about 21 to 23 microns formed into a web having a basis weight of about grams per square meter and a density of about 0.13 grams per cubic centimeter. The fabric may be surface treated with about 0.3 weight percent of a surfactant, such as a surfactant commercially available from Hodgson Textile Chemicals, Inc. under the trade designation AHCOVEL Base N-62. The surfactant may be applied by any conventional means, such as spraying, printing, brush coating or similar techniques. The surfactant may be applied to the entire bodyside liner 44 or may be selectively applied to particular sections of the bodyside liner 44, such as the medial section along the longitudinal centerline of the diaper, to provide greater wettability of such sections. The bodyside liner may further include a lotion or treatment applied thereto that is configured to be transferred to the wearer's skin. Suitable compositions for application to the bodyside liner 44 are described in U.S. Pat. No. 6,149,934 that issued to Krzysik et al. on Nov. 21, 2000.

The representative absorbent articles of the invention can include an absorbent core 28 disposed between the outer cover 42 and the bodyside liner 44. The absorbent core 28 of the diaper/diaper-pant 20, as representatively illustrated in FIG. 2 and FIG. 4, may suitably include a matrix of hydrophilic fibers, such as a web of cellulosic fluff, mixed with particles of a high-absorbency material commonly known as superabsorbent material. In a particular aspect, the absorbent core 28 includes a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent core 28 may include a laminate of fibrous webs and superabsorbent material or other suitable matrix for maintaining a superabsorbent material in a localized area.

The absorbent core 28 may have any of a number of shapes. For example, the absorbent core 28 may be rectangular, I-shaped, or T-shaped. It is generally preferred that the absorbent core 28 is narrower in the intermediate section than in the front or rear waist sections of the diaper 20. The absorbent core 28 may be provided by a single layer or, in the alternative, may be provided by multiple layers, all of which need not extend the entire length and width of the absorbent core 28. In a particular aspect of the invention, the absorbent core 28 can be generally T-shaped with the laterally extending cross-bar of the “T” generally corresponding to the front waist region 22 of the absorbent article for improved performance, especially for male infants.

The size and the absorbent capacity of absorbent core 28 should be compatible with the size of the intended wearer and the liquid loading imparted by the intended use of the absorbent article. Further, the size and the absorbent capacity of the absorbent core 28 can be varied to accommodate wearers ranging from infants through adults. In addition, it has been found that with the present invention, the densities and/or basis weights of the absorbent core 28 can be varied.

The high-absorbency material may be selected from natural, synthetic, and modified natural polymers and materials. The high-absorbency materials may be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent core 28 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful in the present invention.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. In general, the high absorbency material is present in the absorbent core 28 in an amount of from about 5 to about 90 percent by weight, desirably in an amount of at least about 30 percent by weight, and even more desirably in an amount of at least about 50 percent by weight based on a total weight of the absorbent core 28. For example, in a particular aspect, the absorbent core 28 may include a laminate which includes at least about 50 percent by weight and desirably at least about 70 percent by weight of high-absorbency material overwrapped by a fibrous web or other suitable material for maintaining the high-absorbency material in a localized area.

An example of high-absorbency material suitable for use in the present invention is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

Optionally, a substantially hydrophilic tissue or nonwoven wrapsheet (not illustrated) may be employed to help maintain the integrity of the structure of the absorbent core 28. The wrapsheet is typically placed about the absorbent core 28. The wrapsheet may be composed of an absorbent cellulosic material, such as creped wadding or a high wet-strength tissue. In one aspect of the invention, the wrapsheet may be configured to provide a wicking layer that helps to rapidly distribute liquid over the mass of absorbent fibers constituting the absorbent core 28.

Due to the thinness of absorbent core 28 and the high absorbency material within the absorbent core 28, the liquid uptake rates of the absorbent core 28, by itself, may be too low, or may not be adequately sustained over multiple insults of liquid into the absorbent core 28. To improve the overall liquid uptake and air exchange, the diaper/diaper-pant 20 of the different aspects of the present invention may further include a porous, liquid-permeable layer of surge management material 53, as representatively illustrated in FIG. 2 and FIG. 4. The surge management layer 53 is typically less hydrophilic than the absorbent core 28, and has an operable level of density and basis weight to quickly collect and temporarily hold liquid surges, to transport the liquid from its initial entrance point and to substantially completely release the liquid to other parts of the absorbent core 28. This configuration can help prevent the liquid from pooling and collecting on the portion of the diaper/diaper-pant 20 positioned against the wearer's skin, thereby reducing the feeling of wetness by the wearer. The structure of the surge management layer 53 also generally enhances the air exchange within the diaper/diaper-pant 20.

Various woven and nonwoven fabrics can be used to construct the surge management layer 53. For example, the surge management layer 53 may be a layer composed of a meltblown or spunbond web of synthetic fibers, such as polyolefin fibers. The surge management layer 53 may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge management layer 53 may be composed of a substantially hydrophobic material, and the hydrophobic material may optionally be treated with a surfactant or otherwise processed to impart a desired level of wettability and hydrophilicity. In a particular aspect, the surge management layer 53 includes a hydrophobic, nonwoven material having a basis weight of from about 30 to about 120 grams per square meter.

The absorbent articles of the invention can include additional components. For example, as representatively illustrated in FIGS. 1-4, the disposable diaper/diaper-pant 20 may include a pair of containment flaps 56 that are configured to provide a barrier to the lateral flow of body exudates. The containment flaps 56 may be located along the laterally opposed side edges 30 of the diaper/diaper-pant adjacent the side edges of the absorbent core 28. Each containment flap 56 typically defines an unattached edge that is configured to maintain an upright, perpendicular configuration in at least the crotch region 26 of the diaper/diaper-pant 20 to form a seal against the wearer's body. The containment flaps 56 may extend longitudinally along the entire length of the absorbent core 28 or may only extend partially along the length of the absorbent core 28. When the containment flaps 56 are shorter in length than the absorbent core 28, the containment flaps 56 can be selectively positioned anywhere along the side edges 30 of diaper/diaper-pant 20 in the crotch region 26. In a particular aspect of the invention, the containment flaps 56 extend along the entire length of the absorbent core 28 to better contain the body exudates. Such containment flaps 56 are generally well known to those skilled in the art.

The diaper/diaper-pant 20 of the different configurations of the present invention may further include elastics at the waist edges 32 and side edges 30 of the diaper/diaper-pant 20 to further prevent leakage of body exudates and support the absorbent core 28. For example, as representatively illustrated in FIGS. 1-4, the diaper/diaper-pant 20 of the present invention may include a pair of leg elastic members 54 that are connected to the laterally opposed side edges 30 of the diaper/diaper-pant 20 in the crotch region 26. The diaper/diaper-pant 20 may also include a pair of waist elastic members 58 that are connected to the longitudinally opposed waist edges 32 of the diaper/diaper-pant 20. The leg elastics 54 and waist elastics 58 are generally adapted to fit about the legs and waist of a wearer in use to maintain a positive, contacting relationship with the wearer to effectively reduce or eliminate the leakage of body exudates from the diaper/diaper-pant 20.

Materials suitable for use as the leg elastics 54 and waist elastics 58 are well known to those skilled in the art. Exemplary of such materials are sheets or strands or ribbons of a polymeric, elastomeric material that may be adhered to the outer cover 42 in a stretched position, or that may be attached to the outer cover 42 while the outer cover is pleated, such that elastic constrictive forces are imparted to the outer cover 42. The leg elastics 54 may also include such materials as polyurethane, synthetic and natural rubber. The waist elastics 58 may be formed by elastic strands attached to the outer cover 42 or they may be formed by attaching separate pieces of stretchable materials to the waist regions of the article. For example, the waist elastics 58 may include a piece of stretch-bonded laminate material attached to the interior surface 34 of the article to form a waistband. Elasticity may be added or incorporated into the waist opening of absorbent articles utilizing a variety of known approaches. The absorbent articles of the invention may include one or more components that extend laterally outward from the longitudinal sides of the article. Typically, the longitudinal sides are defined by the materials forming the chassis of the diaper/diaper-pant 20. The chassis may be defined by the outer cover 42 and bodyside liner 44 materials. Components that extend laterally outward may include front ear portions 64 and back ear portions 62. The front ear portions 64 and the back ear portions 62 may be formed of one or more materials and may include laminates of materials. The front ear portions 64 and the back ear portions 62 improve the fit of the absorbent article. More specifically, the front ear portions 64 may provide additional coverage around the waist of the wearer and they may assist caregivers with positioning the front waist region 22 on the wearer of the article. The front ear portions 64 may also include mechanical fastening materials such that the front ear portions 64 contribute to the overall fastening system of the article. The back ear portions 62 may also provide coverage around the waist of the wearer. More specifically, the back ear portions 62 may provide the bridging material between the back waist region 24 of the article and the front waist region 22 such that the back ear portions 62 form part of the article's waist opening and an upper edge of the article's leg openings. Additionally, the back ear portions 62 may include fastening materials that facilitate joining of the back waist region 24 with the front waist region 22. For example, the back ear portions 62 may include fasteners 60 selected for engagement with an attachment panel 66 in the front waist region 22 of the article.

Presently available infant diapers typically include back ear portions 62 that include a stretchable material. When the back ear portions 62 include a stretchable material, the back ear portions 62 may increase the range with which the fasteners 60 may be engaged into the attachment panel 66 or directly into the outer cover 42. Further, when the back ear portions 62 include a stretchable material, the article may be worn by a greater range of users as a result of the increased fit range. An exemplary material from which the back ear portions 62 may be constructed is a necked bonded laminate material having two nonwoven (e.g. spunbond) facings with an elastomeric film (e.g. KRATON film) laminated in between. Other suitable stretchable materials are known in the art. Depending on the design of the article, it may also be desirable for the front ear portions 64 to include a stretchable material.

When the product form of the absorbent article is a training pant or a swim pant, the back ear portions 62 and the front ear portions 64 are understood to include the side panels that are attached to the longitudinal sides 30 of the article and also are attached to each other to form side seams of the article. Typically, the side panels of training pants and swim pants are made from stretchable materials. The side panels' ability to stretch allows these products to be pulled on the wearer like underpants.

The diaper-pant 20 form of the present invention (representatively illustrated in FIG. 3 and FIG. 4) may be described as a hybrid between an infant diaper that is typically removed and applied while the child is lying down and a training pant that is put on like underpants. The product form may be referred to as a diaper-pant because the diaper-pant may be applied and removed as either a diaper or a pant. A diaper-pant may have a back ear portion 62 and a front ear portion 64 where the back ear portion 62 and front ear portion 64 are attached to each other by a passive side bond 74. The passive side bond 74 may be selected to be readily tearable by caregivers during the process of “converting” the diaper-pant from a pant to a diaper. Desirably, the passive side bond 74 is easily opened/broken without tearing of the materials used to form the back ear portion 62 and the front ear portion 64.

With each of the product forms, the back ear portions 62 and the front ear portions 64 may be attached to the longitudinal side edges 30 of the article by bonding techniques, such as ultrasonic bonding. Use of ultrasonic bonding techniques tends to form discrete bond points. The bond patterns of the invention, as will be discussed herein, may be formed using known techniques such as adhesive, thermal, laser and pressure that are capable of forming the patterns. Ultrasonic bonding will be referred to for purposes of example. With articles of the invention, the back ear portions 62 and the front ear portions 64 may be attached to one or more of the chassis materials using one or more bond patterns. The articles of the invention may include an attachment area 76 where the attachment area 76 includes an overlapping area of the material forming the outer cover 42 and the material forming either a back ear portion 62 or a front ear portion 64. The attachment area 76 may further include a variegated bond pattern 70 and a uniform bond pattern 72 where each bond pattern provides attachment between the outer cover 42 material and the ear portion material. The attachment area 76 may also include an overlapping area of the material forming the bodyside liner 44 and the material forming either a back ear portion 62 or a front ear portion 64. Additionally, the attachment area 76 may include an overlapping area of the material forming the outer cover 42, the material forming the bodyside liner 44 and the material forming either a back ear portion 62 or a front ear portion 64.

The variegated bond pattern 70 may be formed by a plurality of bond points that are variable in location with respect to each other. Put differently, the bond points of the variegated bond pattern 70 may not all be located equidistantly from each other. While the bond points of the variegated bond pattern 70 may spaced irregularly with respect to each other, the variegated bond pattern 70 may have a repeating pattern appearance (as illustrated by the variegated bond pattern 70 shown in FIG. 7). Examples of suitable variegated bond patterns 70 are illustrated in FIGS. 6-7. The uniform bond pattern 72 may be formed by a plurality of bond points that are regular in location with respect to each other. Put differently, the bond points of the uniform bond pattern 72 are located generally equidistantly from each other. Examples of suitable uniform bond patterns 72 are also illustrated in FIGS. 6-7. The variegated bond pattern 70 and the uniform bond pattern 72 may be immediately adjacent to each other as illustrated in FIG. 6 or the patterns may have some open space in between them as illustrated in FIG. 7. The bond patterns may be formed by ultrasonic bonds or by other bonding techniques capable of providing individual bond points.

The combination of a variegated bond pattern 70 and a uniform bond pattern 72 for attaching ear portion material to outer cover material and/or liner material provides several advantages. For example, the variegated bond pattern 70 is configured to preferentially fail before the uniform bond pattern 72 when a lateral force is applied to the ear portion 62, 64. Desirably, the variegated bond pattern 70 is run as close as possible to the longitudinal side edge 30 of the outer cover 42 material or the bodyside liner 44 material. The benefits of the variegated bond pattern 70 failing preferentially may be discerned when stretchable materials are selected for the ear portion 62, 64 material and materials having a longitudinal orientation are selected for the outer cover 42 and/or bodyside liner 44 materials. In this case, when a user or caregiver of the diaper/diaper-pant 20 pulls laterally outward on an ear portion 62, 64, the stretching of the ear portion 62, 64 material may result in the failure of the variegated bond pattern 70 but will prevent the outer cover 42 and/or bodyside liner 44 materials from easily tearing in the longitudinal direction 38. Usage of a combination of a variegated bond pattern 70 and a uniform bond pattern 72 helps to minimize the amount of ear portion 62, 64 material that is in the attachment area 76, therefore making more stretchable ear portion 62, 64 material available for article wrap (coverage around the wearer). Additional stretchable ear portion 62, 64 material also becomes available when the variegated bond pattern 70 fails. Consequently, the combination of the variegated bond pattern 70 and the uniform bond pattern 72 provides more secure attachment of diaper/diaper-pant 20 components while maximizing the functional benefits provided by the materials selected to form the components.

Both the variegated bond pattern 70 and the uniform bond pattern 72 may include individual bond points having similar properties. The bond points may be regularly-shaped (e.g. circular, rectangular, triangular, square, polygon, etc.) or irregularly-shaped. The bond points may have an area of from about 0.00017 square inches (in²) to about 0.0032 square inches (in²). For circular bond points, this range of areas would correspond to bond point diameters of about 0.015 inches to about 0.055 inches. The area of a bond point may be determined using techniques known to those of skill in the art. For example, the diameter of a circular bond point may be measured by placing the bond pattern under a microscope, such as a SWIFT microscope model #3208. The measurement may be made at about 4+ magnification. A lens that may be used is a SWIFT POINTMASTER ocular lens (EW 10×D/20.50 14.5 MM). This type of lens is a scaled ocular lens that may be calibrated to a graduated slide with a 2 mm measurement divided into units of 0.01 mm. Such a slide is manufactured by American Optical of Southbridge, MA. The calibration indicated that 67 gradations equaled 2 mm.

The individual bond points forming the variegated bond pattern 70 and the uniform bond pattern 72 may be characterized by their bond angle. The individual bond points may have a bond angle of from about 5 degrees to about 25 degrees. Determination of the bond angle is described within U.S. Pat. No. 5,733,411 issued to Bett on Mar. 31, 1998, for example at Col. 10, lines 50-59 (U.S. Pat. No. 5,733,411 is incorporated herein by reference thereto).

The variegated bond pattern 70 and the uniform bond pattern 72 may be in close lateral proximity to each other or they may be spaced somewhat apart. For example, the variegated bond pattern 70 and the uniform bond pattern 72 may be laterally spaced apart from each other by about 2 millimeters. The bond patterns may be spaced further apart depending on the product configuration with which they are being used. Additionally, the variegated bond pattern 70 and the uniform bond pattern 72 may have a combined width in the lateral direction of from about 8 to about 25 millimeters. This combined width in the lateral direction therefore represents the width in the lateral direction 40 of the attachment area 76. As described herein and as illustrated in the figures, the variegated bond pattern 70 may be located laterally outward of the uniform bond pattern 72.

In order to provide the improved attachment of diaper/diaper-pant 20 components, the bond patterns of the invention may have different bond strengths. The bond strength may be represented by a bond thickness that may be “normalized” to account for the bonding together of different materials (e.g. spunbond material, film material, necked bonded laminate material, point unbonded material, etc.). Heavier basis weight materials will have thicker bond spots and lighter basis weight materials will have thinner bond spots proportional to their basis weights. The “normalized” thicknesses are comparable between bonded materials of different basis weights. The difference between the thickness of the variegated bond pattern 70 and the thickness of the uniform bond pattern 72 may be characterized as a normalized bond thickness differential. The normalized bond thickness differential of the bond patterns may be from about 3.7 millimeters/gram/square centimeters (mm/g/cm²) to about 13.6 mm/g/cm². Strong bonds have a normalized bond thickness of from about 3.8 to about 6.3 mm/g/cm². Bonds having good strength but also consuming less energy to form have a normalized bond thickness of from about 6.3 to about 9.8 mm/g/cm².

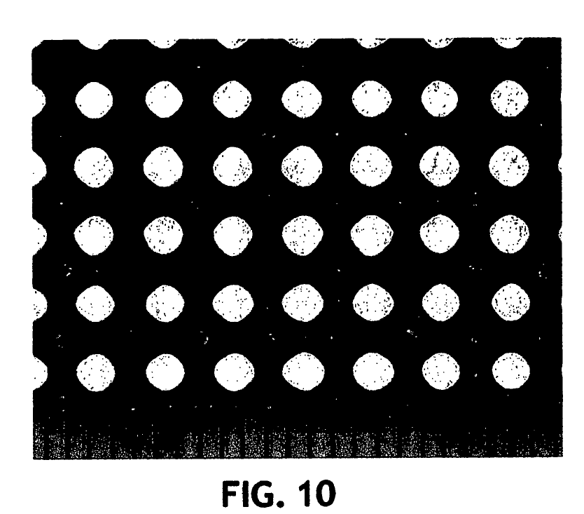

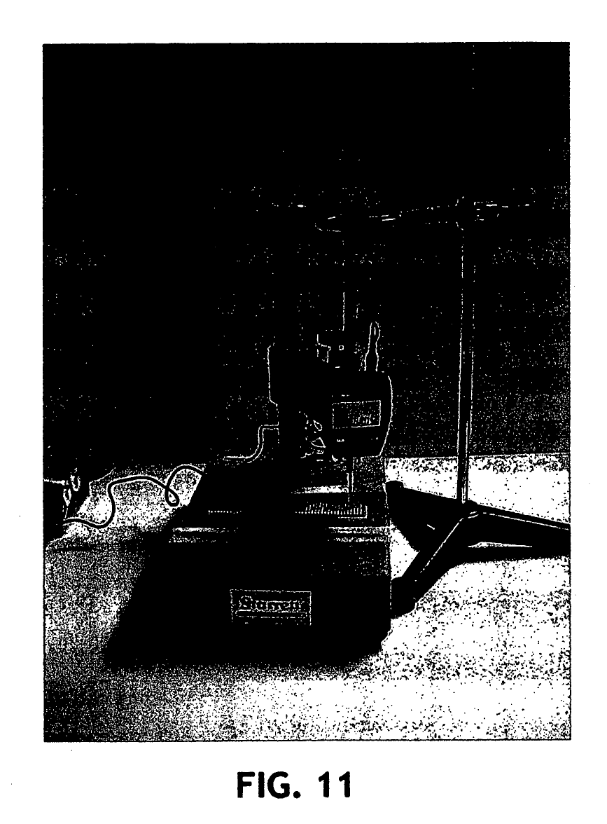

The normalized bond thickness differential was determined by identifying the normalized thickness of bonds that do not separate (i.e. the bonded material(s) tear before the bond fails) and subtracting the normalized thickness of bonds that separate very easily (low strength). In order to determine the point at which bond strength changes from “strong” (bonded materials tear before bond fails) to “weak” (bond easily separates), a series of bonds of different strengths may be created and then tested. For example, bonds of varying strength may be made by simulating a variation in pin height; to simulate such a variation, bonds may be formed by changing the gap between the sonic bonding horn (Branson 920 plunge bonder) and the bond pattern. Larger gaps between the horn and the bond pattern represent shorter pins and smaller gaps between the horn and the bond pattern represent taller pins. The “taller” pins will make the bonds more compact and thinner, resulting in increased bond strength; the “shorter” pins will make bonds that are less compact and thicker. The bond pattern illustrated In the photograph of FIG. 10 was used to form bonds of varying “gaps” (simulating variance in pin height) between a necked bonded laminate material (basis weight of 111.2 grams/square meter; used to form back ear portions on commercially-available HUGGIES Convertibles diaper-pants sold by Kimberly-Clark Corporation) and a point unbonded material (basis weight of 71.2 grams/square meter; used to form front ear portions on commercially-available HUGGIES Convertibles diaper-pants sold by Kimberly-Clark Corporation). The average bond thicknesses were measured using a Mitutoyo Model ID F125E digital thickness measurement gauge and the strength of the bonds was measured according to ASTM test procedure D-5733-99 (“Trap Tear Test”; modified to not put a starter cut in the sample). In order to accurately measure the thickness of the bonds (using the Mitutoyo digital thickness measurement gauge), a flat-topped pin having a diameter equal to or less than the diameter of the bond point was placed underneath the bond point. Additionally, the flat-topped pin had a height equal to or greater than the thickness of the materials that were bonded together. The rounded end (about 5 mm in diameter) of the Mitutoyo digital thickness measurement gauge was centered within the top of the bond point. The Mitutoyo digital thickness measurement gauge had an additional 990 gram (approximate) weight on top of it in order to provide approximately 990 grams of force to minimize variability. The set-up that was used for measuring bond thickness is shown in the photograph provided as FIG. 11. A series of eight different bonds were created and measured for their thickness and strength; the results are provided in Table 1. below.

TABLE 1

| | | Bond Thickness | | |
|---|---|---|---|---|
| | Average | Normalized for | Tear | Visual |
| | Bond | Basis Weight of | Strength | Characterization |
| Sample | Thickness | Materials | (average | of Separation |
| Number | (mm) | (mm/g/cm²) | peak load) | During Testing |
| 1 | 0.068 | 3.70 | 2534 | NBL tore |
| 2 | 0.089 | 4.87 | 2228 | NBL tore |
| 3 | 0.121 | 6.63 | 2520 | NBL tore |
| 4 | 0.163 | 8.93 | 2551 | NBL tore |
| 5 | 0.180 | 9.84 | 2554 | NBL tore |
| 6 | 0.211 | 11.56 | 325 | No Tearing |
| 7 | 0.220 | 12.04 | 1799 | No Tearing |
| 8 | 0.249 | 13.63 | 112 | No Tearing |

The “average” bond thickness was determined by making three to four thickness measurements of three different bonds for each sample. Desirably, the difference between the normalized thickness of the variegated bond pattern 70 and the normalized thickness of the uniform bond pattern 72 is between 3.7 and 13.6 mm/g/cm². The benefit provided by this differential is that the variegated bond pattern 70 will preferentially fail before the uniform bond pattern 72.

In addition to providing attachment between the ear portions 62, 64 and the longitudinal side edge 30 of the diaper/diaper-pant 20, the bond patterns of the invention may also provide for the attachment of fasteners 60 to the laterally outward edges of ear portions 62, 64. For example, the ear portion 62, 64 may include a fastener attachment area 61 (as shown in FIG. 9) where the fastener attachment area 61 includes an overlapping area of the ear portion 62, 64 material and the fastener 60 material. The fastener attachment area 61 may include a low density bond pattern 78 and a high density bond pattern 80. The low density bond pattern 78 may be distinguished from the high density bond pattern 80 by a difference in the percent of bonded area. The percent bonded area may be defined as the cumulative area of the individual bond points divided by the area covered by the bond pattern (e.g. a portion of the fastener attachment area 61). The bond pattern area may be defined by the product of the bond pattern width and the bond pattern length. The area of individual bond points may be determined as previously described herein. The low density bond pattern 78 may have a percent bond area of about 9% to about 18% and the high density bond pattern 80 may have a percent bond area of about 25%.

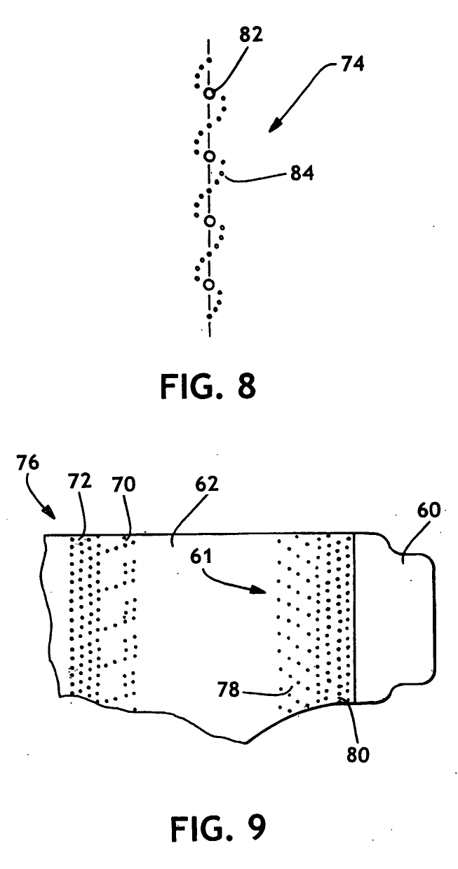

Combinations of bond patterns may also be used to improve the attachment between a back ear portion 62 and a front ear portion 60 to form a passive side bond 74. The passive side bond 74 may be formed by a short distance bond pattern 84 and a long distance bond pattern 82 as illustrated in FIG. 8. The short distance bond pattern 84 may have a smaller bond area than the long distance bond pattern 82. For example, the long distance bond pattern 82 may include individual bond points that have areas of from about 1.7 mm² (square millimeters) to about 28 mm² (square millimeters). The short distance bond pattern 84 may include individual bond points that have areas of from about 0.75 mm² to about 3 mm². The bond points of the long distance bond pattern 82 may be spaced apart from each other by at least 0.5 inches. The individual bond points of the short distance bond pattern 84 may be spaced apart from each other by less than 0.5 inches. The benefits of combining a long distance bond pattern 82 and a short distance bond pattern 84 include improved opening of the passive side bond 74 with reduced tearing. Typically, the bonds of the long distance bond pattern 82 are of greater strength than the bonds of the short distance bond pattern 84. When a wearer or caregiver opens the passive side bond 74 of an article of the invention, it is expected that holes would form around the larger, higher strength bonds but that the bond points of the short distance bond pattern 84 would open easily and without tearing. The bond points of the short distance bond pattern 84 may provide sufficient attachment of the ear materials without causing tearing of those materials upon opening of the passive side bond 74. The differential in bond strength between the long distance bond pattern 82 and the short distance bond pattern 84 contributes to these advantages. In another example of the differences between the long distance bond pattern 82 and the short distance bond pattern 84, there may be a normalized bond thickness differential between the two bond patterns of from about 3.7 to about 13.6 mm/g/cm².

While the bond patterns of the invention have been described in detail with respect to specific aspects thereof, it will be appreciated that those skilled in the art, upon attaining an understanding of the foregoing, may readily conceive of alterations to, variations of and equivalents to these bond patterns. Accordingly, the scope of the present invention should be assessed as that of the appended claims and any equivalents thereto.

We claim:

1. A disposable absorbent article including a longitudinal direction and a lateral direction, further comprising:
an outer cover, wherein the outer cover includes a longitudinal edge and wherein the outer cover includes an outer cover material;
an ear portion, wherein the ear portion extends laterally outward from the longitudinal edge of the outer cover and wherein the ear portion includes an ear portion material; and
an attachment area, wherein the attachment area includes an overlapping area of the outer cover material and the ear portion material and wherein the attachment area includes a variegated bond pattern and a uniform bond pattern.

2. The disposable absorbent article of claim 1, wherein the outer cover material includes a laminate of a nonwoven material and a film material.

3. The disposable absorbent article of claim 2, wherein the film material is extensible.

4. The disposable absorbent article of claim 1, wherein the ear portion material includes a stretchable material.

5. The disposable absorbent article of claim 1, wherein the variegated bond pattern includes individual bond points having an area of from 0.00017 in² to 0.0032 in².

6. The disposable absorbent article of claim 1, wherein the variegated bond pattern includes individual bond points having a bond angle of from 5 to 25 degrees.

7. The disposable absorbent article of claim 1, wherein the uniform bond pattern includes individual bond points having an area of from 0.00017 in² to 0.0032 in².

8. The disposable absorbent article of claim 1, wherein the uniform bond pattern includes individual bond points having a bond angle of from 5 to 25 degrees.

9. The disposable absorbent article of claim 1, wherein the variegated bond pattern and the uniform bond pattern have a lateral spacing from each other of from 2 mm to 10 mm.

10. The disposable absorbent article of claim 1, wherein the variegated bond pattern and the uniform bond pattern have a combined width in the lateral direction of from 8 to 25 mm.

11. The disposable absorbent article of claim 1, wherein the variegated bond pattern is located laterally outward from the uniform bond pattern.

12. The disposable absorbent article of claim 1, wherein a difference between a thickness of the variegated bond pattern and a thickness of the uniform bond pattern is a normalized bond thickness differential of from 3.7 to 13.6 mm/g/cm².

13. The disposable absorbent article of claim 1, wherein the ear portion further includes a fastener attachment area, wherein the fastener attachment area includes an overlapping area of the ear portion material and a fastener material and wherein the fastener attachment area includes a low density bond pattern and a high density bond pattern.

14. The disposable absorbent article of claim 13, wherein the low density bond pattern has a percent bond area of 9% to 18% and the high density bond pattern has a percent bond area of 25%.

15. A disposable absorbent article including a longitudinal direction and a lateral direction, further comprising:
a bodyside liner, wherein the bodyside liner includes a longitudinal edge and wherein the bodyside liner includes a bodyside liner material;
an ear portion, wherein the ear portion extends laterally outward from the longitudinal edge of the bodyside liner and wherein the ear portion includes an ear portion material; and
an attachment area, wherein the attachment area includes an overlapping area of the bodyside liner material and the ear portion material and wherein the attachment area includes a variegated bond pattern and a uniform bond pattern.

16. The disposable absorbent article of claim 15, wherein the ear portion material includes a stretchable material.

17. The disposable absorbent article of claim 15, wherein the variegated bond pattern includes individual bond points having an area of from 0.00017 in² to 0.0032 in².

18. The disposable absorbent article of claim 15, wherein the variegated bond pattern includes individual bond points having a bond angle of from 5 to 25 degrees.

19. The disposable absorbent article of claim 15, wherein the uniform bond pattern includes individual bond points having an area of from 0.00017 in² to 0.0032 in².

20. The disposable absorbent article of claim 15, wherein the uniform bond pattern includes individual bond points having a bond angle of from 5 to 25 degrees.

21. The disposable absorbent article of claim 15, wherein the variegated bond pattern and the uniform bond pattern have a lateral spacing of from 2 mm to 10 mm.

22. The disposable absorbent article of claim 15, wherein the variegated bond pattern and the uniform bond pattern have a combined width in the lateral direction of from 8 to 25 mm.

23. The disposable absorbent article of claim 15, wherein the variegated bond pattern is located laterally outward from the uniform bond pattern.

24. The disposable absorbent article of claim 15, wherein a difference between a thickness of the variegated bond pattern and a thickness of the uniform bond pattern is a normalized bond thickness differential of from 3.7 to 13.6 mm/g/cm².

25. The disposable absorbent article of claim 15, wherein the ear portion further includes a fastener attachment area, wherein the fastener attachment area includes an overlapping area of the ear portion material and a fastener material and wherein the fastener attachment area includes a low density bond pattern and a high density bond pattern.

26. The disposable absorbent article of claim 25, wherein the low density bond pattern has a percent bond area of 9% to 18% and the high density bond pattern has a percent bond area of 25%.

27. A disposable absorbent article including a longitudinal direction and a lateral direction, further comprising:
an outer cover, wherein the outer cover includes a longitudinal edge and wherein the outer cover includes an outer cover material;
a bodyside liner in overlapping relation to the outer cover, wherein the bodyside liner includes a longitudinal edge and wherein the bodyside liner includes a bodyside liner material;
an ear portion, wherein the ear portion extends laterally outward from the longitudinal edge of the outer cover and wherein the ear portion includes an ear portion material; and
an attachment area, wherein the attachment area includes an overlapping area of the outer cover material, the bodyside liner material and the ear portion material and wherein the attachment area includes a variegated bond pattern and a uniform bond pattern.

28. A disposable absorbent article including a longitudinal direction and a lateral direction, further comprising:
a chassis, wherein the chassis includes a front waist region, a back waist region and a crotch region intermediate the front waist region and the back waist region;
a back ear portion, wherein the back ear portion is attached to the back waist region;
a front ear portion, wherein the front ear portion is attached to the front waist region; and
a side bond between the back ear portion and the front ear portion, wherein the side bond includes a short distance bond pattern and a long distance bond pattern.

29. The disposable absorbent article of claim 28, wherein the short distance bond pattern has a smaller bonded area than the long distance bond pattern.

30. The disposable absorbent article of claim 28, wherein the long distance bond pattern includes individual bond points having an area of from 1.7 to 28 mm².

31. The disposable absorbent article of claim 28, wherein the long distance bond pattern includes individual bond points spaced apart from each other by at least 0.5 inches.

32. The disposable absorbent article of claim 28, wherein the short distance bond pattern includes individual bond points having an area of from 0.75 to 3 mm².

33. The disposable absorbent article of claim 28, wherein the short distance bond pattern includes individual bond points spaced apart from each other by no greater than 0.5 inches.

34. The disposable absorbent article of claim 28, wherein a difference between a thickness of the long distance bond pattern and a thickness of the short distance bond pattern is a normalized bond thickness differential of from 3.7 to 13.6 mm/g/cm².

35. A disposable absorbent article including a longitudinal direction and a lateral direction, further comprising:
a chassis, wherein the chassis includes a front waist region, a back waist region and a crotch region intermediate the front waist region and the back waist region;
a back ear portion, wherein the back ear portion is attached to the back waist region;
a front ear portion, wherein the front ear portion is attached to the front waist region; and
a side bond between the back ear portion and the front ear portion, wherein the side bond includes a variegated bond pattern and a uniform bond pattern.

36. The disposable absorbent article of claim 35, wherein a difference between a thickness of the variegated bond pattern and a thickness of the uniform bond pattern is a normalized bond thickness differential of from 3.7 to 13.6 mm/g/cm².
